(19) United States
(12) Patent Application Publication
(10) Pub. No.: US 2022/0218719 A1
(43) Pub. Date: Jul. 14, 2022

(54) MATRIX COMPOSITION COMPRISING (S)-5-BENZYL-N-(5-METHYL-4-OXO-2,3,4,5-TETRAHYDROBENZO[B][1,4]OXAZEPIN-3-YL)-4H-1,2,4-TRIAZOLE-3-CARBOXAMIDE

(71) Applicant: GLAXOSMITHKLINE INTELLECTUAL PROPERTY DEVELOPMENT LIMITED, Brentford, Middlesex (GB)

(72) Inventors: Rachel APFELBAUM, Collegeville, PA (US); Xin CHEN, Collegeville, PA (US); Rennan PAN, Collegeville, PA (US)

(21) Appl. No.: 17/610,531

(22) PCT Filed: May 15, 2020
(86) PCT No.: PCT/IB2020/054615

§ 371 Date: Nov. 11, 2021

Related U.S. Application Data
(60) Provisional application No. 62/990,093, filed on Mar. 16, 2020.
(60) Provisional application No. 62/849,180, filed on May 17, 2019.

(51) Int. Cl. A61K 31/553; A61K 9/20; A61P 17/06

(52) U.S. Cl. CPC A61K 31/553; A61K 9/2018; A61P 17/06; A61K 9/2077; A61K 9/2054

(57) ABSTRACT

The present invention relates to modified release pharmaceutical compositions of a RIP1 kinase inhibitor compound and forms thereof, processes or methods of composition preparation and uses or treatment methods therefor.

### FIELD OF THE INVENTION

The present invention relates to modified release pharmaceutical compositions of a RIP1 kinase inhibitor compound and forms thereof, processes or methods of composition preparation and uses or treatment methods therefor.

### BACKGROUND OF THE INVENTION

RIP1 kinase-dependent programmed necrosis is rapidly emerging as a key pathogenic mechanism in a wide variety of inflammatory diseases, especially those driven by TNFα. (S)-5-benzyl-N-(5-methyl-4-oxo-2,3,4,5-tetrahydrobenzo[b][1,4]oxazepin-3-yl)-4H-1,2,4-triazole-3-carboxamide (Compound 1 or GSK2982772), or a tautomer thereof, or a salt thereof, is a highly selective inhibitor of RIPK1. In pK/pD studies, Compound 1 concentrations in plasma/blood declined rapidly until approximately 12 hours post dose, with a t_{1/2} of approximately 2-3 hours and a high ratio of Cₘₐₓ/Cₘᵢₙ using a 120 mg dose of an immediate release (IR) formulation. Weisel et al., Pharmacol Res Perspect. 2017; e00365, https://doi.org/10.1002/prp2.365; page 10.

In the case of the Compound 1 IR formulation, it was reported that in a pharmacokinetics study of 120 mg strength IR formulation in 13 subjects, the geometric mean Cₘₐₓ and C_{24 h} were 1.375 and 0.006 μg/mL respectively, with a Cₘₐₓ:C_{24 h} ratio of 280.

Once daily (QD) administration with no food restrictions is considered optimal when designing oral drug products. QD is the most common oral dosing regimen and most preferred by patients. Once daily administration facilitates compliance and reduces medication management issues and improves therapeutic outcomes. Twice daily dosing is also well accepted by most patients. Oral dosing regimens that require more than twice daily administration are associated with poorer adherence. In addition to dose frequency and number of dosing, dosing restrictions also play an important role. In particular, the need for co-administration with food or administration requiring the avoidance of food for a particular period of time greatly increases the complexity of managing polypharmacy and non-adherence.

Therefore, QD administration of Compound 1, with no food restrictions would be preferred. In order to achieve QD dosing, Compound 1 may need to be administered in a manner that achieves a higher blood concentration and/or a larger area under curve (AUC). Increasing the dose and/or increasing the frequency of dosing might be needed. Unfortunately, increasing the IR dose for Compound 1 may lead to toxicity/adverse effects and increasing frequency of dosing may lead to reduced patient adherence. Hence, development of a formulation that could achieve QD dosing, other than an IR formulation, was desired. Applicants describe herein a controlled-release or modified-release (MR) formulation for Compound 1.

In developing a MR composition, which includes traditional polymer matrices, such as a hypromellose matrix and more complex multilayered matrices with or without the addition of functional excipients (used by the pharmaceutical technology industry to describe an excipient that can provide an added function or quality over and above the “conventional” excipients (http://www.pharmtech.com/excipients-formulation-success), a number of approaches have been disclosed. The use of a coating to control the rate of release of an active agent has received considerable attention and many different devices have been developed for that purpose.

One particular MR approach was developed and commercialized by GlaxoSmithKline under the tradename of DiffCORE. The approach involves using mechanical drilling of film-coated tablets, e.g., enteric-coated tablets, to form apertures of specific size, number and position in the film coat. The release rate of the drug can be modified and controlled through altering the exposed surface area, the composition of the tablet core, altering the number of apertures and/or altering the position of the apertures. The manufacture of tablets uses standard manufacturing unit operations such as blending, granulation and compression.

In developing a QD formulation of Compound 1, because of its short half-life, and the associated drug delivery issues, the inventors developed the present invention which is directed to overcoming these and other problems encountered.

### SUMMARY OF THE INVENTION

In one aspect, the invention provides a modified release pharmaceutical composition comprising an erodible matrix core, which core comprises (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, or a salt thereof.

In a second aspect, the present invention provides a modified release pharmaceutical composition comprising an erodible matrix core, which core comprises a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, or a salt thereof and at least one component which is a release modifier, the erodible matrix core having a film coating around said core.

In another aspect, the invention provides a modified release pharmaceutical composition comprising an erodible matrix core, which core comprises a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, or a salt thereof and at least one component which is a release modifier, the erodible matrix core having a film coating around said core, which film coating comprises at least one erodible material and at least one aperture in the film coating.

In one aspect, the invention provides a modified release pharmaceutical composition comprising an erodible matrix core, which core comprises 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof.

In a second aspect, the invention provides a modified release pharmaceutical composition comprising an erodible matrix core, which core comprises 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, and at least one component which is a release modifier, the erodible matrix core having a film coating around said core.

In another aspect, the invention provides a modified release pharmaceutical composition comprising an erodible matrix core, which core comprises 480 mg of a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, and at least one component which is a release modifier, the erodible matrix core having a film coating around said core, which film coating comprises at least one erodible material and at least one aperture in the film coating.

In one embodiment of the invention, the modified release pharmaceutical composition comprises 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, and at least one component which is a release modifier, the erodible matrix core having a film coating around said core, which film coating comprises at least one erodible material and at least one aperture in the film coating, and the at least one aperture in the film coating is of a pre-determined size.

In one aspect, the invention provides a modified release pharmaceutical composition comprising an erodible matrix core, which core comprises 480 mg of a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, for once daily dosing.

In a second aspect, the invention provides a modified release pharmaceutical composition comprising an erodible matrix core, which core comprises 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, and at least one component which is a release modifier, the erodible matrix core having a film coating around said core, for once daily dosing.

In another aspect, the invention provides a modified release pharmaceutical composition comprising an erodible matrix core, which core comprises 480 mg of a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film coating comprises at least one erodible material and at least one aperture in the film coating, for once daily dosing.

In one embodiment of the invention, the modified release pharmaceutical composition, for once daily dosing, comprises 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, and the least one aperture in the film coating is of a pre-determined size.

In one embodiment of the invention, the modified release pharmaceutical composition is an oral dosage form.

In one embodiment of the invention, the modified release pharmaceutical composition is an oral dosage form, which oral dosage form is a tablet.

In one embodiment of the invention, the modified release pharmaceutical composition is an oral dosage form, which oral dosage form is a tablet which is suitable for once daily dosing.

In one embodiment of the invention, the modified release composition is administered once daily as two tablets, each tablet comprising 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film coating comprises at least one erodible material and at least one aperture in the film coating.

In one aspect, the invention provides a tablet comprising an erodible matrix core, which core comprises:

(a) about 30 mg to about 480 mg of a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, or a salt thereof;

(b) about 1.0% wt/wt to about 60% wt/wt of a release modifier;

(c) about 1.0% wt/wt % to about 95% wt/wt of a diluent;

(d) about 0.25% wt/wt to about 5.0% wt/wt of a lubricant; and

(e) a film coating around said erodible matrix core comprising an erodible material in an amount of about 1.0% wt/wt of the tablet core and about 10% wt/wt of the tablet core, and at least one aperture in the film coating.

In one aspect, the invention provides a tablet comprising an erodible matrix core, which core comprises:

(a) about 240 mg to about 480 mg of a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, or a salt thereof;

(b) about 1.0% wt/wt to about 20% wt/wt of a release modifier;

(c) about 15% wt/wt % to about 30% wt/wt of a diluent;

(d) about 0.50% wt/wt to about 3.0% wt/wt of a lubricant; and

(e) a film coating around said erodible matrix core comprising an erodible material in an amount of about 2.0% wt/wt of the tablet core and about 6% wt/wt of the tablet core, and at least one aperture in the film coating.

In one aspect, the invention provides a tablet comprising an erodible matrix core, which core comprises:

(a) about 480 mg of a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, or a salt thereof;

(b) about 20% wt/wt of a release modifier;

(c) about 25% wt/wt of a diluent;

(d) about 1.0% wt/wt of a lubricant; and

(e) a film coating around said erodible matrix core comprising an erodible material in an amount of about 4% wt/wt of the tablet core, and at least one aperture in the film coating.

In one aspect, the invention provides a method of treating a subject with psoriasis comprising administering to said subject a therapeutically effective amount of a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, wherein said compound is administered in modified release pharmaceutical composition comprising an erodible matrix core, which core comprises (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof.

In a second aspect, the invention provides a method of treating a subject with psoriasis comprising administering to said subject a therapeutically effective amount of a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, wherein said compound is administered in a modified release pharmaceutical composition comprising an erodible matrix core, which core comprises (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core.

In another aspect, the invention provides a method of treating a subject with psoriasis comprising administering to said subject a therapeutically effective amount of a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, wherein said compound is administered in a modified release pharmaceutical composition comprising an erodible matrix core, which core comprises (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film-coating comprises at least one erodible material and at least one aperture in the film coating.

In yet another aspect, the invention provides a method of treating a subject with psoriasis comprising administering to said subject a modified release pharmaceutical composition in the form of a tablet, said tablet comprising an erodible matrix core, which core comprises, 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film coating comprises at least one erodible material and at least one aperture in the film coating, and wherein two of said tablets are administered once daily.

In one embodiment of the invention, the subject is a mammal. In one embodiment of the invention, the subject is a human.

In one aspect, the invention provides a modified release pharmaceutical composition comprising an erodible matrix core, which core comprises (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, for use in treating psoriasis.

In a second aspect, the invention provides a modified release pharmaceutical composition comprising an erodible matrix core, which core comprises (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, for use in treating psoriasis.

In another aspect, the invention provides a modified release pharmaceutical composition comprising an erodible matrix core, which core comprises (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film-coating comprises at least one erodible material and at least one aperture in the film coating, for use in treating psoriasis.

In yet another aspect, the invention provides a modified release pharmaceutical composition comprising an erodible matrix core, which core comprises 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film coating comprises at least one erodible material and at least one aperture in the film coating, for use in treating psoriasis.

In one aspect, the invention provides a modified release pharmaceutical composition comprising an erodible matrix core, which core comprises 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, in the manufacture of a medicament for use in the treatment of psoriasis.

In a second aspect, the invention provides a modified release pharmaceutical composition comprising an erodible matrix core, which core comprises 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, in the manufacture of a medicament for use in the treatment of psoriasis.

In another aspect, the invention provides a modified release pharmaceutical composition comprising an erodible matrix core, which core comprises 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film-coating comprises at least one erodible material and at least one aperture in the film coating, in the manufacture of a medicament for use in the treatment of psoriasis.

In any of the above-described aspects or embodiments of the invention, the modified release pharmaceutical composition is useful in treating moderate psoriasis.

In any of the above-described aspects or embodiments of the invention, the modified release pharmaceutical composition is useful in treating severe psoriasis.

In any of the above-described aspects or embodiments of the invention, the modified release pharmaceutical composition is useful in treating moderate to severe psoriasis.

In any one of the above-described aspects or embodiments of the invention, the modified release pharmaceutical composition is an oral dosage form.

In any one of the above-described aspects or embodiments of the invention, the modified release pharmaceutical composition is an oral dosage form, which oral dosage form is a tablet.

In any one of the above-described aspects or embodiments of the invention, the dosage form is a tablet having a front face and a back face. In one embodiment of the invention, there is an aperture on the front face of the dosage form. In one embodiment of the invention, there is an aperture on the back face of the dosage form. In one embodiment of the invention, there is an aperture on the front face of the dosage form and, there is an aperture on the back face of the dosage form.

In any one of the above-described aspects or embodiments of the invention, the at least one aperture creates an exposed surface area on the dosage form.

In any one of the above-described aspects or embodiments of the invention, the at least one aperture in the film coating is of a pre-determined size.

In any one of the above aspects or embodiments of the invention, the release rate of the compound, or tautomer thereof, is partially modified by altering the size of the at least one aperture in the film coating.

In any one of the above-described aspects or embodiments of the invention, the release rate of the compound, or tautomer thereof, is partially modified by altering the number of the apertures in the film coating.

In any one of the above-described aspects or embodiments of the invention, the release rate of the compound, or tautomer thereof, is partially modified by altering the size of the aperture in the film coating and altering the number of apertures in the film coating.

In any one of the above-described aspects or embodiments of the invention, the release rate of the compound, or tautomer thereof, is partially modified by altering the size of the aperture in the film coating or altering the number of apertures in the film coating.

In any of these above-described embodiments of the invention, the modified release pharmaceutical composition comprises a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof.

In any of the above-described embodiments of the invention, the modified release pharmaceutical composition comprises 480 mg of a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof.

In any of these above-described embodiments of the invention, the modified release pharmaceutical composition is in the form of a tablet, said tablet comprising 480 mg of a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, wherein two of such modified release tablets are administered to a subject, once daily. It will be understood that the dosage is therefore 960 mg per day.

In any of these above-described embodiments of the invention, the modified release pharmaceutical composition is in the form of a tablet, said tablet comprising an erodible matrix core, which core comprises 480 mg of a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film coating comprises at least one erodible material and at least one aperture in the film coating, and wherein two of such modified release tablets are administered once daily. It will be understood that the dosage is therefore 960 mg per day.

### BRIEF DESCRIPTION OF THE DRAWINGS

FIG. 1 depicts the dissolution profiles of two Minitablet modified release formulations, one with 20% w/w of Methocel K15M, and the other with 60% w/w of Methocel K15M in 0.1 N HCl with USPII.

FIG. 2 depicts the mean concentration time plots for the IR 120 mg tablet, the Minitabs modified release 8 h 120 mg tablet (fasted), and the Minitabs modified release 12 h 120 mg (fasted and standard fed); linear (FIG. 2A) and semi-log (FIG. 2B) scales.

FIG. 3 depicts the mean concentration-time profile for Minitabs modified release 120 mg (fastest and slowest release) and immediate release 120 mg; linear (FIG. 3A) and semi-log (FIG. 3B) scales.

FIG. 4 depicts mean plasma GSK2982772 concentration-time (μg/ml) plot by formulation: IR versus 240 mg DiffCORE MR-12 h, MR-16 h and MR-18 h, fasted Part A study 209261.

FIG. 5 depicts a dosing curve of Compound 1 (GSK2982772) 240 mg tablet, DiffCORE composition vs. IR composition.

FIG. 6 depicts a side-by-side comparison of the performance of a prototype Minitabs modified release formulation and DiffCORE MR in fasted and fed states both compared to an IR reference performance.

### DETAILED DESCRIPTION OF THE INVENTION

GSK2982772 is a first-in-class, highly selective, receptor-interacting protein-1 (RIP1) kinase inhibitor being developed for the treatment of a disease or disorder selected from inflammatory bowel disease, Crohn's disease, ulcerative colitis, psoriasis, retinal detachment, retinitis pigmentosa, macular degeneration, pancreatitis, atopic dermatitis, rheumatoid arthritis, spondylarthritis, gout, juvenile idiopathic arthritis, systemic-onset juvenile idiopathic arthritis (SoJIA), systemic lupus erythematosus, Sjogren's syndrome, systemic scleroderma, anti-phospholipid syndrome, vasculitis, osteoarthritis, non-alcohol steatohepatitis, alcohol steatohepatitis, autoimmune hepatitis autoimmune hepatobiliary diseases, primary sclerosing cholangitis, nephritis, Celiac disease, autoimmune ITP, transplant rejection, ischemia reperfusion injury of solid organs, sepsis, systemic inflammatory response syndrome, cerebrovascular accident, myocardial infarction, Huntington's disease, Alzheimer's disease, Parkinson's disease, allergic diseases, asthma, atopic dermatitis, multiple sclerosis, type I diabetes, Wegener's granulomatosis, pulmonary sarcoidosis, Bechet's disease, interleukin-1 converting enzyme associated fever syndrome, chronic obstructive pulmonary disease, tumor necrosis factor receptor-associated periodic syndrome, and periodontitis.

In one embodiment of the invention, the disease or disorder is selected from inflammatory bowel disease, Crohn's disease, ulcerative colitis and psoriasis. In one embodiment of the invention, the disease is inflammatory bowel disease. In one embodiment of the invention, the disease is Crohn's disease. In one embodiment of the invention, the disease is ulcerative colitis. In one embodiment of the invention, the disease is psoriasis. In one embodiment of the invention, the disease is mild to moderate psoriasis. In one embodiment of the invention, the disease is moderate to severe psoriasis.

The compound of the invention, (S)-5-benzyl-N-(5-methyl-4-oxo-2,3,4,5-tetrahydrobenzo[b][1,4]oxazepin-3-yl)-4H-1,2,4-triazole-3-carboxamide, has a structure which is:

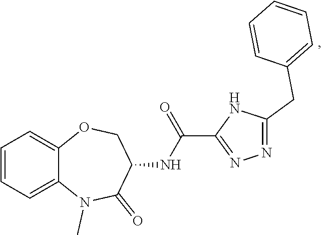 [SMILES: CN1C(=O)[C@@H](NC(=O)c2nnc(Cc3ccccc3)[nH]2)COc2ccccc21]

or a tautomer thereof. In one embodiment, the invention provides a modified release pharmaceutical composition comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, as described in WO 2014/125444A1.

In a second embodiment, the invention provides a modified release pharmaceutical composition comprising an erodible matrix core, which core comprises a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core.

In a third embodiment, the invention provides a modified release pharmaceutical composition comprising an erodible matrix core, which core comprises a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film coating comprises at least one erodible material and at least one aperture in the film coating.

In a fourth embodiment, the invention provides a modified release pharmaceutical composition comprising an erodible matrix core, which core comprises a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film coating comprises at least one erodible material and at least one aperture in the film coating, and wherein the at least one aperture in the film coating is of pre-determined size.

In a fifth embodiment, the invention provides a modified release pharmaceutical composition comprising an erodible matrix core, which core comprises a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film coating comprises at least one erodible material and at least one aperture in the film coating, wherein the release rate of the compound, or tautomer thereof, is partially modified by altering the size of the at least one aperture in the film coating, and wherein the compound is present in the composition in a crystalline form.

In a sixth embodiment, the invention provides a modified release pharmaceutical composition comprising an erodible matrix core, which core comprises a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film coating comprises at least one erodible material and at least one aperture in the film coating, wherein the release rate of the compound, or tautomer thereof, is partially modified by altering the number of apertures in the film coating, and wherein the compound is present in the composition in a crystalline form.

In a seventh embodiment, the invention provides a modified release pharmaceutical composition comprising an erodible matrix core, which core comprises a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film coating comprises at least one erodible material and at least one aperture in the film coating, wherein the release rate of the compound, or tautomer thereof, is partially modified by altering the size of the at least one aperture in the film coating and altering the number of apertures in the film coating, and wherein the compound is present in the composition in a crystalline form.

In one aspect, the invention provides a method of treating a subject with psoriasis comprising administering to said subject, a pharmaceutical composition as described above, taken as two 480 mg tablets which provide a total of 960 mg of Compound 1, once daily, each tablet comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof.

For chronic inflammatory conditions, a QD dosing option would offer greater convenience, and hence potentially optimize compliance. Compound 1, having a half-life or 2-3 hours, was not envisioned to be capable of once daily dosing. Nonetheless, the modified release pharmaceutical composition of this invention is a once daily dosage form for Compound 1. It is the modification of the release rate of Compound 1 via the composition as described herein, which enables once daily dosing.

Multiple clinical trials were conducted to evaluate the safety and efficacy profiles of Compound 1 for the potential treatment of inflammatory diseases, including psoriasis, rheumatoid arthritis (RA) and ulcerative colitis (UC).

As used herein, the term “aperture” is synonymous with the terms hole, orifice, passageway, outlet, and the like.

As used herein, the terms “fasted state”, “fasted” or “fasted dosing” are interchangeable and refer to clinical trial studies wherein participants were provided a light snack the evening before dosing and then refrained from food and drink (except water) for a minimum of 10 h before dosing to approximately 4 h after dosing.

As used herein, the term “high-fat meal” refers to clinical trial studies wherein the participants eat a meal consisting of one fried egg, two strips of bacon, two slices of buttered bread, one hash brown, and 240 mL of whole milk.

As used herein, the terms “standard meal” or “standard dosing” are used interchangeable and refer to clinical trial studies wherein the participants eat 50 g cereal with 240 mL of whole milk, one croissant, and one small pot of jam. The standard meal commenced 30 min prior to dosing and completed approximately 5 min before dosing. At least 90% of the meal had to be consumed for dosing to proceed.

The formulations used in early clinical studies included a 0.1 mg/mL oral solution to support doses up to 5 mg; active-pharmaceutical-ingredient (API) in HPMC capsules for doses between 5 mg to 120 mg; and roller-compacted IR tablets of 10, 20 and 30 mg strength.

The pharmacokinetic (PK) profile of Compound 1 when administered as an immediate release (IR) formulation was characterized by rapid absorption and elimination with time to maximum concentration (Tₘₐₓ) of approximately 2 hours post-dose. After attaining a maximum blood plasma concentration (Cₘₐₓ), blood plasma concentrations of Compound 1 declined rapidly until approximately 12 hours post-dose, with an elimination half-life (t_{1/2}) of 2-3 hours followed by a slower terminal phase t_{1/2} of approximately 5-6 hours. A majority of the systemic exposure to GSK2982772 was associated with the shorter half-life of 2-3 hours.

The short half-life demonstrated that in order to achieve maintained target efficacious concentrations, frequent dosing was needed. The IR formulation of GSK2982772 was also dosed twice daily (BID) and thrice daily (TID). For chronic inflammatory conditions, a QD dosing option would offer greater convenience and potentially optimize compliance. The following studies were conducted to evaluate the feasibility of developing a QD modified-release (MR) formulation as well as to evaluate the impact of food on selected formulations.

A mini-tablet modified release formulation (“Minitabs MR” or “MT”) was also tested in a clinical setting. For the Minitabs MR formulation, the target range of in vitro drug release duration was 80% of drug dissolved over 10 h for “fast” release and 80% of drug dissolved over 24 h for “slow” release. GSK2982772 was administered as 120 mg Minitabs MR at the slowest (MT-12 h; 80% release at 12 h) and fastest (MT-8 h; 80% release at 8 h) release rates which represented the extremes of the achieved design space. It was originally planned that a slower release rate of 80% over 24 h would be evaluated, however, because of a size restriction of the Minitabs MR formulation, it was not possible to increase the polymer content beyond 60% w/w hypromellose 2208, and therefore a release rate of slower than 80% release at 12 h was not achievable with Minitabs MR.

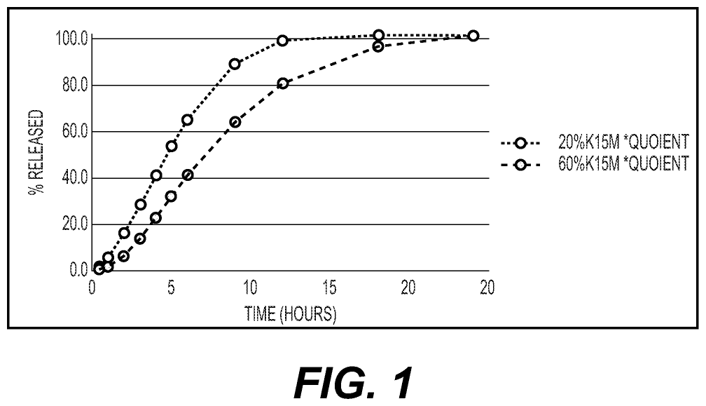

Each Minitabs MR contained 5 mg of GSK2982772 (total weight 20 mg) in an HPMC capsule, and a single polymer, which was adjusted between 20% and 60% weight by weight (w/w). As shown in FIG. 1, different tablet release/dissolution rates of the Minitabs MR were achieved by varying the Methocel K15M polymer level between 60% and low 20% in the formulation. Minitabs MR composition details can be found in Table 1.

TABLE 1
Composition of GSK2982772 Minitab
(IMPD Submission Study 205017)

| | 60% Polymer | | 20% Polymer | |
|---|---|---|---|---|
| | Uncoated | | Uncoated | |
| | Minitabs | | Minitabs | |
| | %/w/w | mg/tab | %/w/w | mg/tab |
| GSK2982772A | 25.00 | 5.00 | 25.00 | 5.00 |
| Methocel K15M Premium CR | 60.00 | 12.00 | 20.00 | 4.00 |
| Mannitol | 10.00 | 2.00 | 10.00 | 2.00 |
| Avicel PH 102 | 4.00 | 0.80 | 44.00 | 8.80 |
| Magnesium Stearate | 1.00 | 0.20 | 1.00 | 0.20 |
| Tablet Core | 100.00 | 20.00 | 100.00 | 20.00 |

Part A (N=19) of Study ID 205017 evaluated pharmacokinetics following administration of GSK2982772 Minitab MR formulation with a fixed sequence of 120 mg single doses of 80% release at 12 hours (MT-12 h) or 8 hours (MT-8 h), immediate-release tablet (all fasted), and MT-12 h (high-fat meal). Part B (N=10) evaluated once-daily dosing for 3 days with MT-12 h: 120 mg (fasted), 240 mg (fasted), and 300 mg (standard meal). See Example 5 for more details of the clinical trial study.

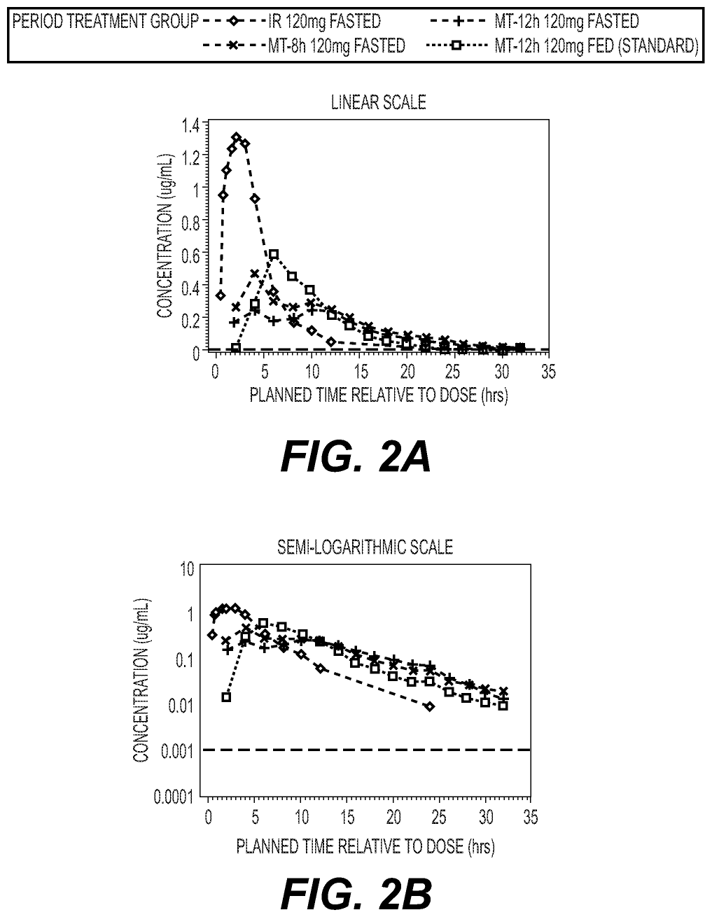

The pharmacokinetics evaluation of the Minitabs MR and IR shows sustained release was achieved with the Minitab MR formulation (as illustrated in FIG. 2; FIG. 2A depicts the data on a linear scale and FIG. 2B depicts the data on a semi-logarithmic scale). All modified-release formulations reduced maximum plasma concentration (Cₘₐₓ), delayed time to Cₘₐₓ, and increased 24 h concentration vs. immediate-release. Bioavailability relative to immediate-release was 61% for MT-12 h and 73% for MT-8 h. Dosing MT-12 h with a high-fat meal increased Cₘₐₓ 2.25-fold and area under the plasma concentration vs. time curve (0-inf) 1.24-fold compared with the fasted state. Three days of once-daily MT-12 h led to minimal accumulation of GSK2982772.

In Part A, there was a rank order relationship between the duration of drug release and the PK of GSK2982772 with the 120-mg Minitab MR formulations. The longer release duration of Minitab MR-12 h resulted in longer Tₘₐₓ, lower Cₘₐₓ, higher C₂₄ₕ, and reduced AUC_{0-inf} compared to Minitab MR-8 h. The overall extent of absorption was lower with the Minitab MR formulations compared with the IR (73% for MT-8 h and 61% for MT-12 h).

MT-12 h was selected to study the effect of food. Both rate and extent of absorption of GSK2982772 were increased when MT-12 h was administered with a high-fat meal compared with the fasted state. Cₘₐₓ was more than doubled (2.25-fold increase), Cₘₐₓ:Cₘᵢₙ ratio was increased by approximately five-fold, and overall extent of absorption was approximately 25% higher.

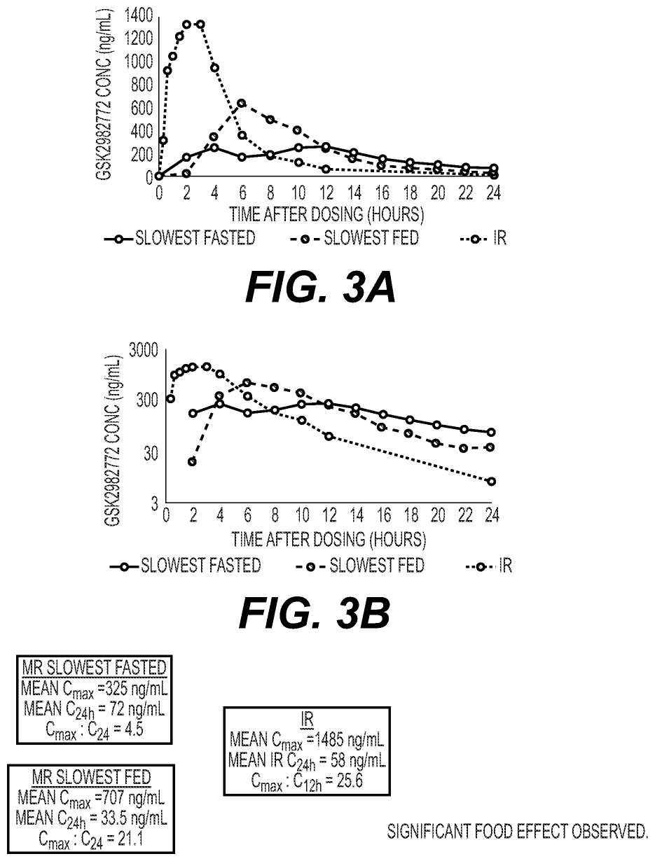

A food effect with a high-fat meal was observed with the Minitabs MR (See FIG. 3; FIG. 3A depicts the data on a linear scale and FIG. 3B depicts the data on a semi-logarithmic scale). The Minitabs MR-12 h formulation when administered with a high-fat meal increased both Cₘₐₓ and AUC. While the Minitabs MR formulations provided flatter pharmacokinetic profiles vs. the immediate-release formulation, the Minitabs MR formulations was susceptible to a food effect with a high-fat meal.

A subsequent clinical trial study (Study ID 209261; see Example 6) was conducted using the 240 mg DiffCORE modified release (DiffCORE MR) technology of the instant invention (also referred to as GSK2982772 MR formulation) specifically utilizing a combination of a controlled-release polymer matrix core (the “erodible matrix core”) and controlled-release erodible coating, which was an enteric coating. Dosing with all 240 mg DiffCORE MR tablet formulations (MR-12 h, MR-16 h and MR-18 h) resulted in flatter PK profiles, with a delay to Tₘₐₓ, a reduction in Cₘₐₓ and AUC, and an increase in C₂₄ₕ (concentrations at 24 h) compared to the IR 240 mg formulation. The time nomenclature of 12 h, 16 h and 18 h for DiffCORE MR-12 h, DiffCORE MR-16 h and DiffCORE MR-18 h, respectively, represent the approximate time for 80% of GSK2982772 to be released in an in-vitro dissolution test. Dissolution testing was conducted using a USPII apparatus, in a two-stage dissolution test. Conditions were 0.01N HCl, 0.4% CTAB for one hour, and then pH 6.8 phosphate buffer with 0.4% CTAB. Paddle speed was 100 rpm.

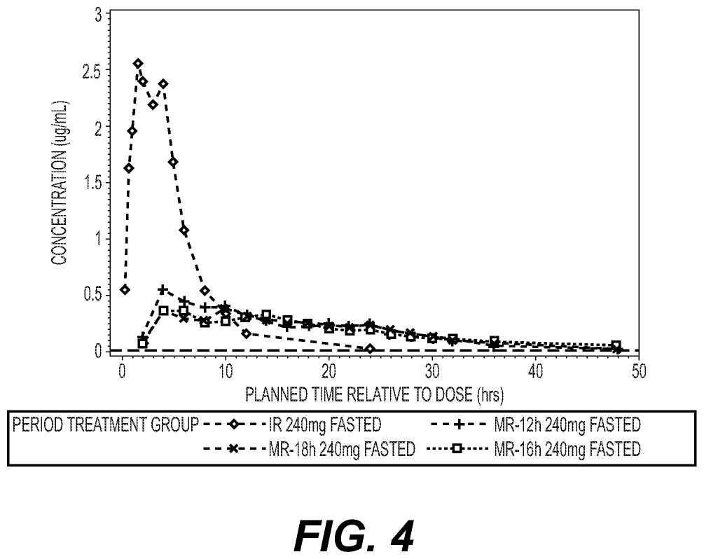

Median Tₘₐₓ increased with increasing 240 mg DiffCORE MR release rate. Cₘₐₓ, AUC and C₂₄ₕ values were similar across the range of 240 mg DiffCORE MR release rates. Specifically, in the fasted state, Tₘₐₓ was prolonged by 4 h relative to IR; Cₘₐₓ was reduced to approximately 14% of IR Cₘₐₓ; and C₂₄ₕ was ˜10-fold higher than for IR. The bioavailability of DiffCORE MR relative to IR based on AUC_{(0-inf)} ranged from approximately 50% to 60% in the fasted state. The bioavailability of GSK2982772 for DiffCORE MR-16 h was 48% relative to IR. See FIG. 4.

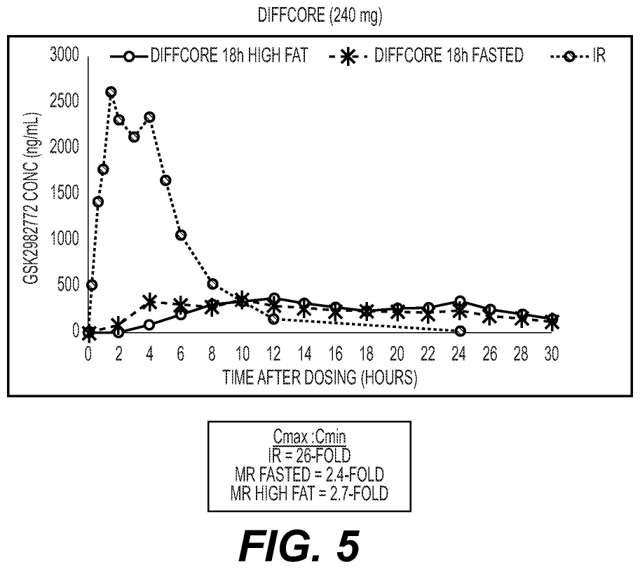

Following administration of 240 mg DiffCORE MR with a high fat meal, there was no notable impact of food on Cₘₐₓ or AUC relative to the fasted state. See FIG. 4 and FIG. 5. The DiffCORE MR formulation with an erodible core enabled once a day dosing for a compound having a half-life of approximately 2-3 hours.

Suitably, the invention provides a modified release tablet of the invention comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, wherein, upon administration to a human, produces a total drug exposure across time value (AUC) of Compound 1, which is about 30% to about 55% lower when compared to the total drug exposure across time value of Compound 1 produced by an instant release tablet. In one embodiment of the invention, the modified release tablet comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, upon administration to a human, produces a total drug exposure across time value of Compound 1, which is about 30% to about 50% lower when compared to the total drug exposure across time value of Compound 1, produced by an instant release tablet.

Suitably, the invention provides a modified release tablet of the invention comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, wherein, upon administration to a human, produces a maximum blood plasma concentration of Compound 1, which is about 70% to about 90% lower when compared to the maximum blood plasma concentration of Compound 1 produced by an instant release tablet. In one embodiment of the invention, the modified release tablet comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, upon administration to a human, produces a maximum blood plasma concentration of Compound 1, which is about 75% to about 90% lower when compared to the maximum blood plasma concentration of Compound 1 produced by an instant release tablet. In another embodiment of the invention, the modified release tablet comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, upon administration to a human, produces a maximum blood plasma concentration of Compound 1, which is about 80% to 90% lower when compared to the maximum blood plasma concentration of Compound 1 produced by an instant release tablet.

Suitably, the invention provides a modified release tablet of the invention comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, wherein, upon administration to a human, produces a blood plasma concentration at 24 hours (C₂₄) of Compound 1 which is about 10-fold to about 25-fold lower when compared to the blood plasma concentration at 24 hours of Compound 1 produced by an instant release tablet. In one embodiment of the invention, a modified release tablet of the invention comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, upon administration to a human, produces a blood plasma concentration at 24 hours (C₂₄) of Compound 1 which is about 12-fold to about 18-fold lower when compared to the blood plasma concentration at 24 hours of Compound 1 produced by an instant release tablet. In one embodiment of the invention, a modified release tablet of the invention comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, upon administration to a human, produces a blood plasma concentration at 24 hours (C₂₄) of Compound 1 which is about 15-fold to about 18-fold lower when compared to the blood plasma concentration at 24 hours of Compound 1 produced by an instant release tablet. In one embodiment of the invention, a modified release tablet of the invention comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, upon administration to a human, produces a blood plasma concentration at 24 hours (C₂₄) of Compound 1 which is about 10-fold lower when compared to the blood plasma concentration at 24 hours of Compound 1 produced by an instant release tablet.

Suitably, the invention provides a modified release tablet of the invention comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, wherein, upon administration to a human, produces a time to maximum concentration value (Tₘₐₓ) of Compound 1 which is about 3 to about 4 hours greater than the time to maximum concentration value of Compound 1 produced by an instant release tablet. In one embodiment of the invention, the modified release tablet of the invention comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, upon administration to a human, produces a time to maximum concentration value of Compound 1 which is about 3 hours greater than the time to maximum concentration value of Compound 1 produced by an instant release tablet. In one embodiment of the invention, the modified release tablet of the invention comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, upon administration to a human, produces a time to maximum concentration value of Compound 1 which is about 4 hours greater than the time to maximum concentration value of Compound 1 produced by an instant release tablet.

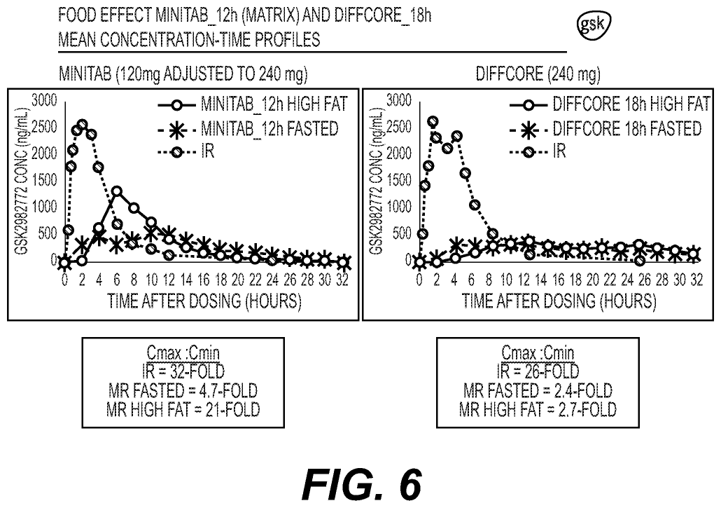

A side-by-side comparison of the mean concentration-time profiles of the Minitabs MR formulation and a 240 mg DiffCORE MR tablet depicts the food effect observed with the Minitabs MR formulation and the lack of a notable impact of food on Cₘₐₓ or AUC relative to the fasted state using the DiffCORE MR 240 mg with a high fat meal. See FIG. 6.

Following administration of 480 mg DiffCORE MR-16 h with a standard meal, median Tₘₐₓ was slightly delayed compared to the fasted state and there was no notable impact on Cₘₐₓ, or AUC_{(0-inf)}, with geometric mean ratios of 1.02 and 0.97, respectively.

In one aspect, the invention provides a modified release pharmaceutical composition comprising an erodible matrix core, which core comprises (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, or a salt thereof.

In a second aspect, the present invention provides a modified release pharmaceutical composition comprising an erodible matrix core, which core comprises a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, or a salt thereof and at least one component which is a release modifier, the erodible matrix core having a film coating around said core.

In one embodiment of the invention, the compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, is a free base.

In one embodiment of the invention, the modified release pharmaceutical composition comprises a crystalline form of the free base compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof.

In one embodiment of the invention, the modified release pharmaceutical composition comprises at least one component which is a release modifier, within the erodible matrix core. Release modifying polymers which may be water swellable or not, include, inter alia, cross-linked sodium carboxymethylcellulose, cross-linked hydroxypropylcellulose, hydroxyethylcellulose, hydroxypropylmethylcellulose, polyethylene oxide, polyacrylic acid, carboxymethyl amide, potassium methacrylate divinylbenzene co-polymer, polymethyl-methacrylate, cross-linked polyvinylpyrrolidone, hydroxyethyl cellulose, high-molecular weight polyvinyl alcohols, and the like. Release modifying gellable polymers include methylcellulose, carboxymethylcellulose, low-molecular weight hydroxypropylmethyl cellulose, hydroxyethyl cellulose, low-molecular weight polyvinyl alcohols, polyoxyethylene glycols, non-cross linked polyvinylpyrrolidone, xanthan gum, and the like. Release modifying polymers simultaneously possessing swelling and gelling properties include medium-viscosity hydroxypropylmethylcellulose and medium-viscosity polyvinyl alcohols.

In one embodiment of the invention, the release modifier is a cellulose polymer. In another embodiment of the invention, the release modifier is a hypromellose-hydroxypropylmethyl cellulose polymer. In a further embodiment of the invention, the release modifier is a Methocel polymer. In yet another embodiment, the release modifier is Methocel K3LV. In still another embodiment of the invention, the release modifier is Methocel K100LV. In still yet another embodiment of the invention, the release modifier is Methocel K4M. Suitably, the release modifier is a mixture of cellulose polymers.

Suitably, the release modifier is present in the formulation in an amount between about 1% and about 60% weight/weight of the total composition. In one embodiment of the invention, the release modifier is present in an amount between about 1.0% and about 45% weight/weight of the total composition. In one embodiment of the invention, the release modifier is present in an amount between about 1.0% and about 20% weight/weight of the total composition. In one embodiment of the invention, the release modifier is present in an amount between about 20% and about 45% weight/weight of the total composition. In another embodiment of the invention, the release modifier is present in an amount of about 20% to about 25% weight/weight of the total composition. In another embodiment of the invention, the release modifier is present in an amount of about 20% weight/weight of the total composition. 240 mg DiffCORE MR compositions falling within the scope of the instant invention can be found in Example 1. 60 mg and 480 mg DiffCORE MR compositions falling within the scope of the instant invention can be found in Example 2.

In one aspect, the invention provides a film coating comprises at least one erodible material. In one embodiment of the invention, the erodible material is present in an amount between about 1% wt/wt of the tablet and about 20% wt/wt of the tablet core. In one embodiment of the invention, the erodible material is present in an amount between about 1% wt/wt of the tablet core to about 10% wt/wt of the tablet core. In one embodiment of the invention, the erodible material is present in an amount between about 2% wt/wt of the tablet and about 6% wt/wt of the tablet. In another embodiment of the invention, the erodible material is present in an amount between about 3.6% wt/wt of the tablet core and about 4.6% wt/wt of the tablet core. In one embodiment, the erodible material is present in an amount of about 4% wt/wt of the tablet core. Suitably, the erodible material creates a film coating covering about 4 mg/cm² to about 6 mg/cm² of the tablet surface area. In one embodiment of the invention, the erodible material film coating covers 6 mg/cm² of the tablet surface area.

Suitably, the erodible material of the invention includes various polymethacrylate polymers, co-processed polyvinylacetate phthalate, cellulose acetate trimellitate, cellulose acetate phthalate, shellac, hydroxypropylmethyl cellulose phthalate polymers and their copolymers. The coating material is suitably selected so that it is insoluble or only slightly soluble in stomach acid i.e., at pH 1.5-2, and is soluble or erodible in the small intestine i.e., at around pH 5.5 or in the large intestine i.e., at around pH 7. To achieve this, typically the material of the coating is erodible at pH of 4.5 or above. Suitably, the coating material is selected from: cellulose acetate trimellitate (CAT) dissolving at pH 4.8, polyvinyl acetate phthalate dissolving at pH 5.0, hydroxypropylmethyl cellulose phthalate dissolving at pH 5.2, hydroxypropylmethyl cellulose phthalate dissolving at pH 5.4, Acryl-EZE™ dissolving at pH 5.5, Aquateric™ dissolving at pH 5.8, cellulose acetate phthalate dissolving at pH 6.0, Eudragit™ L30 D dissolving at pH 5.5, Eudragit™ L dissolving at pH 6.0, Eudragit™ S dissolving at pH 6.8, and shellac dissolving at pH 7.2.

In one embodiment of the invention, the erodible material is selected from enteric polymers such as HPMC-AS, poly(meth)acylate polymer, e.g., Acryl-EZE™, and the like. In one embodiment, the erodible material is an enteric coating based on methacrylic acid copolymers such as Eudragit™ polymers. In one embodiment of the invention, the erodible material is an enteric coating which is Eudragit™ L30-D55, Plasacryl.

When necessary, the erodible coating may be modified by addition of plasticizers or anti-tack agents. Suitable materials for this purpose include waxy materials such as glycerides, for example glyceryl monostearate, and the like.

The above reference to the coating being erodible includes the situation where the coating disintegrates partially or wholly, or dissolves, or becomes porous, on contact with an environmental fluid which allows the fluid to contact the core. Suitably the coating disintegrates partially. Suitably the coating disintegrates wholly. Suitably the coating dissolves. Suitably the coating becomes porous.

Similarly, references to the core being erodible includes the situation where core disintegrates partially or wholly, or dissolves, or becomes porous, on contact with an environmental fluid which allows the fluid to contact the active agent. Suitably the core disintegrates partially. Suitably the core disintegrates wholly. Suitably the core dissolves. Suitably the core becomes porous. The core may be a material which allows pH dependent erosion or disintegration of the core to release the compound, or a tautomer thereof from its matrix.

As a protection for the core material, to prevent contamination via the aperture(s) before dosing, it may desirable to provide a conventional seal coating to either the core, or to the dosage form after formation of the aperture(s). The seal coat may be a sub-coat or over-coat to the erodible coating. Suitably, the seal coat is an aqueous film coating. In one embodiment of the invention, the aqueous film coating is present in an amount of between about 1.5% wt/wt and 2.5% wt/wt. In one embodiment of the invention, the aqueous film coating is present is an amount of about 2% wt/wt. In one embodiment of the invention, the aqueous film coating is Opadry® Beige 026170003 is a proprietary film-coating material purchased by GlaxoSmithKline to an agreed specification.

In one aspect, the invention provides a modified release pharmaceutical composition comprising an erodible matrix core, which core comprises a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film coating comprises at least one erodible material and at least one aperture in the film coating, wherein the release rate of the compound, or tautomer thereof, is partially modified by altering the size of the at least one aperture in the film coating.

For the aperture(s) in the coating to retain its integrity and the release rate, the aperture(s) is of a pre-determined size. Typical sizes for the aperture(s), when circular, to be formed in the coating are in the range about 0.5 mm to about 8 mm of diameter, such as 1, 2, 3. 4 or 5 mms in diameter, depending on the overall size of the tablet and the desired rate of release.

In one embodiment of the invention, the at least one aperture is between about 4.0 mm and about 5.5 mm. In one embodiment of the invention, the at least one aperture is between about 4.5 mm and about 5.0 mm. In one embodiment of the invention, the at least one aperture is about 4.5 mm.

Suitably, the oral dosage form has two opposed primary surfaces, conveniently referred to as a front face and a back face. In one embodiment of the invention, there is one aperture on the front face of the oral dosage form which aperture is about 4.5 mm. Where more than one aperture is provided, the apertures may be located on the same face of the oral dosage form, or on opposing faces of the oral dosage form. In one embodiment of the invention, there are two apertures, one on the front face of the dosage form and one on the back face of the dosage form. In one embodiment of the invention, the aperture on the front face of tablet is about 4.5 mm and the aperture on the back face of tablet is about 4.5 mm. Each aperture may be centered on the face of the tablet or off-center.

Suitably, the aperture(s) may have any convenient geometrical shape, for example oval, round, square, but a rounded shape, e.g., substantially circular or elliptical, is generally preferred. More elaborate shapes, such as text characters or graphics, may also be formed, provided that the release rate can be made uniform in individual dosage forms.

Suitably, the apertures(s) may be formed by methods disclosed in U.S. Pat. No. 7,923,027. Typically, aperture(s) may be formed by drilling, for example using mechanical drill bits, ultrasonic cutting or laser beams, or by punches that remove the cut area. The formation of the aperture(s) may by default remove a small portion of the exposed core. It will be understood that the at least one aperture is drilled through the coating, i.e., within the film coating, and just reaches the core but does not penetrate to core.

When a full enteric coated tablet comprising Compound 1 was tested, in some patients the enteric coating disabled any drug release (no efficacy). Typically, for DiffCORE formulations, it is the aperture size and number that controls drug release over time. For the present invention, the release rate of Compound 1 is only partially modified or controlled by the size and/or number of apertures. Without being bound to any particular theory of the invention, by design the film coating herein protects the erodible matrix core from stomach stress, including the pH of the fluid environment. Therefore, only a small amount of drug is released in the stomach through the aperture(s) in the film coating. As the drug is released in the stomach through the aperture(s), the tablet becomes smaller assisting movement of the tablet from the stomach to the intestines. Once in the intestines, the film coating surrounding the erodible matrix core, begins to erode, thereby releasing Compound 1 in a controlled manner over an extended time period. This was unexpected given the short half-life of Compound 1.

So that the aperture(s) in the film coating retains its integrity and control of release rate, it is desirable that the erosion of the coating has a defined pH threshold, i.e., the coating does not substantially erode except in the intestines. Thus, it is envisaged that erosion of the coating has a defined, pre-determined pH threshold at which it dissolves. In one embodiment, the coating erodes at pH greater than about 4.5. In another embodiment, the coating erodes in the pH range from about 4.5 to about 8. In yet another embodiment, the coating erodes in the pH range about 5 to about 7.5.

In applying the concepts of this invention, Compound 1, or a tautomer, or a salt thereof, may be incorporated into a conventional oral tablet or controlled release matrix (including both swellable and non-swellable systems). The matrix is formed into cores which are then coated with an erodible material, for example an erodible coating based on a methacrylic copolymer. One or more openings may then be drilled through the coatings using conventional techniques as disclosed in U.S. Pat. No. 5,004,614.

The present invention finds particular use in the situation where the coating erodes in the pH conditions of the intestines. Accordingly, the present invention also provides an oral dosage form comprising an erodible matrix core which contains a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, the core having a coating with one or more apertures leading to the core, wherein the coating is erodible under the pH conditions prevailing in a mammalian intestine.

It will be appreciated that the use of a coating that erodes at pH>4.5 will restrict the amount of drug released into the acidic conditions associated with the stomach, since release at low pH levels is substantially limited to diffusion of the active agent through the aperture(s) in the erodible coating. As the dosage form leaves a low pH environment, e.g., the stomach, and then encounters a higher environmental pH, e.g., the intestine, the coating will start to dissolve and erode away to expose all of the tablet core. During coat erosion, the available surface area to release drug is increased.

In one aspect, the invention provides a modified release pharmaceutical composition comprising an erodible matrix core, which core comprises a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film coating comprises at least one erodible material and at least one aperture in the film coating, wherein the release rate of the compound, or tautomer thereof, is partially modified by altering the number of apertures in the film coating.

Suitably, there is a range of surface area covered by the aperture(s) in the film coating. In one embodiment of the invention, this range is 1-50% of the tablet surface area. In one embodiment of the invention, the range is 5-15% of the tablet surface area. In one embodiment of the invention, e.g., for a 240 mg tablet having two 4.5 mm apertures, the range is 12.7% of the tablet surface area. It will be understood that by modifying the number of apertures in the film coating, one can partially modify the release rate of the compound.

In a preferred embodiment of the invention, the pharmaceutical composition is an oral tablet comprising a crystalline form of GSK2982772 and a modified release eroding matrix formulation. The tablets are coated with a soluble Opadry® sub-coat and a clear enteric coating of methacrylic acid copolymer. The coated tablets have an aperture drilled through the coating on both faces of the tablet to enable a controlled release of drug in the acidic environment of the stomach.

Suitably, the modified release compositions of the invention may also include diluents/compression aid such as lactose, microcrystalline cellulose, dicalcium phosphate, sucrose, mannitol, xylitol; starches, and the like, and mixtures thereof. In one embodiment of the invention, the diluent is lactose. In one embodiment of the invention, the diluent is lactose monohydrate. In one embodiment of the invention, the diluent is lactose anhydrous. In one embodiment of the invention, the diluent is a combination of lactose monohydrate and lactose anhydrous. In one embodiment of the invention, the diluent is present in an amount of about 1% wt/wt of the tablet core to about 95% wt/wt of the tablet core. In one embodiment of the invention, the diluent is present in an amount of about 1% wt/wt of the tablet core to about 75% wt/wt of the tablet core. In one embodiment of the invention, the diluent is present in an amount of about 10% wt/wt of the tablet core to about 50% wt/wt of the tablet core. In one embodiment of the invention, the diluent is present in an amount of about 15% wt/wt of the tablet core to about 50% wt/wt of the tablet core. In one embodiment of the invention, the diluent is present in an amount of about 15% wt/wt of the tablet core to about 30% wt/wt of the tablet core. In one embodiment of the invention, the diluent is present in an amount of about 20% wt/wt of the tablet core to about 40% wt/wt of the tablet core. In one embodiment of the invention, the diluent is present in an amount of about 20% wt/wt of the tablet core to about 25% wt/wt of the tablet core. In one embodiment of the invention, the diluent is present in an amount of about 25% wt/wt of the tablet core.

Suitably, the modified release compositions of the invention may also include lubricants such as magnesium stearate, sodium stearyl fumarate and stearic acid. In one embodiment of the invention, the lubricant is magnesium stearate. In one embodiment of the invention, a lubricant is present in an amount of about 0.25% wt/wt to about 5.0% wt/wt of the tablet core. In one embodiment of the invention, a lubricant is present in an amount of about 0.50% wt/wt to about 3.0% wt/wt of the tablet core. In one embodiment of the invention, a lubricant is present in an amount of about 0.50% wt/wt to about 1.0% wt/wt of the tablet core. In one embodiment of the invention, a lubricant is present in an amount of about 1.0% wt/wt of the tablet core.

The modified release compositions may further comprise disintegrants, such as crosslinked polyvinylpyrrolidone (CLPVP) and sodium starch glycollate; binders such as povidone (polyvinylpyrrolidone); flow aids such as silicon dioxide or talc.

The core may be prepared by compressing suitable ingredients to form a compacted mass which comprises the core of the dosage form. This may be prepared using conventional excipients and formulation compression methods. Thus, the core typically comprises the active agent or agents along with excipients that impart satisfactory processing and compression characteristics such as diluents, binders and lubricants. Additional excipients that may form part of the core of the device include disintegrants, flavorings, colorants, release modifying agents and/or solubilizing agents such as surfactants, pH modifiers and complexation vehicles.

The core may be produced according to any desired pre-selected shape such as bi-convex, hemi-spherical, near hemi-spherical, round, oval, generally ellipsoidal, oblong, generally cylindrical or polyhedral, e.g., a triangular prism shape. The term “near hemi-spherical” is intended to be construed in the manner described in U.S. Pat. No. 7,923,027.

A suitable manufacturing process for making an oral dosage form of the invention includes transferring to a high shear granulator active component GSK2982772, lactose monohydrate, and hypromellose 2208 K3LV/K100LV/K4M, which components are mixed and granulated with purified water or sterile water for irrigation. The wet mass is then dried and milled. The granules are transferred to a blender. Lactose anhydrous and magnesium stearate are added to the blender. The components are blended. The blend is compressed into tablets and the resultant tablets are coated with an aqueous film coating and an enteric coating. The coated tablets are then drilled on both the front and back tablet faces with a suitably sized aperture, preferably 4.5 mm in size.

The core may be coated with a suitable pH dependent erodible material by any pharmaceutically acceptable coating method. Examples include coating methods disclosed in U.S. Pat. No. 7,923,027 and film coating, sugar coating, spray coating, dip coating, compression coating, electrostatic coating. Typical methods include spraying the coating onto the tablet core in a rotating pan coater or in a fluidized bed coater until the desired coating thickness is achieved. Suitably, the coating is provided to add about 4 to 8 mg/cm2 or 5-7 mg/cm2 of dry polymer around the tablet surface area. Typically, this results in an increase in weight (relative to the core) of from about 3-10% or about 5-10% by weight. Suitably, the coating has a thickness in the range 0.05 to 0.5 mm.

The quantity of Compound 1, or a tautomer thereof, or a salt thereof, present in the core is a “therapeutically effective amount” and means an amount of the compound sufficient to treat the patient's condition but low enough to avoid serious side effects (at a reasonable benefit/risk ratio) within the scope of sound medical judgment. A therapeutically effective amount of the Compound, or a tautomer thereof, or a salt thereof, will vary with the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors and is not limited by the process of this invention.

In one aspect, the invention provides a modified release composition comprising about 30 mg to about 960 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof. In one embodiment of the invention, the modified release composition comprises 30 mg to 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof. In one embodiment of the invention, the modified release composition comprises 30 mg to 240 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof. In one embodiment of the invention, the modified release composition comprises 120 mg to 960 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof. In one embodiment of the invention, the modified release composition comprises 120 mg to 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof. In one embodiment of the invention, the modified release composition comprises 120 mg to 240 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof. In one embodiment of the invention, the modified release composition comprises 480 mg to 960 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof. In one embodiment of the invention, the modified release composition comprises 240 mg to 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof.

In one embodiment of the invention, the modified release composition comprises 120 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof. In one embodiment of the invention, the modified release composition comprises 240 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof. In one embodiment of the invention, the modified release composition comprises 480 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof. In one embodiment of the invention, the modified release composition comprises 960 mg of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof.

In any of these above-described embodiments, the modified release pharmaceutical composition comprises a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof. In one embodiment, Compound 1 is in the form of a free base, or a tautomer thereof.

In any of the above-described embodiments, the modified release pharmaceutical composition comprises 480 mg of a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof.

Suitably, the modified release pharmaceutical composition is administered in a dosage regimen which is sufficient to maintain control over the disorder. In one embodiment of the invention, the dosage regimen is once a day.

In any of these above-described embodiments, the modified release pharmaceutical composition is in the form of a tablet comprising an erodible matrix core, which core comprises 480 mg of a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof and at least one component which is a release modifier, the erodible matrix core having a film coating around said core, which film coating comprises at least one erodible material and at least one aperture in the film coating, and wherein the modified release tablet is administered as two tablets, once daily.

The present invention also extends to formulations which are bioequivalent to the tablets or formulations of the present invention in terms of both rate and extent of absorption, for instance as defined by the US Food and Drug Administration and discussed in the so-called “Orange Book” (Approved Drug Products with Therapeutic Equivalence Evaluations, US Dept of Health and Human Services).

Compositions falling within the scope of the present invention are described in the following Examples. These Examples are intended to be by way of illustration rather than limitation of the present invention

### EXAMPLES

### Example 1: Composition of GSK2982772 240 mg DiffCORE Modified Release Coated Tablets

| | Quantity (mg) per Tablet | | | |
|---|---|---|---|---|
| | 240 mg 50:50 | 240 mg 25:75 | 240 mg 100% | |
| Component | K100:K4M | K100:K4M | K4M | Function |
| Tablet Core | | | | |
| GSK2982772A | 240 | 240 | 240 | Active |
| Hypromellose 2208 (K100LV) | 44.5 | 22.25 | — | Release Modifier |
| Hypromellose 2208 (K4M) | 44.5 | 66.75 | 89.0 | Release Modifier |
| Lactose monohydrate | 71.0 | 71.0 | 71.0 | Diluent |
| Sterile Water for Irrigation | q.s. | q.s. | q.s. | Granulating Fluid |
| Lactose anhydrous | 40.55 | 40.55 | 40.55 | Diluent |
| Magnesium Stearate | 4.45 | 4.45 | 4.45 | Lubricant |
| Tablet Core Weight | 445 | 445 | 445 | |
| Sub Coat | | | | |
| Opadry ® Beige 02B170003 | 11.13 | 11.13 | 11.13 | Film coat |
| Sterile Water for Irrigation | q.s. | q.s. | q.s. | Process aid |
| Enteric Coat | | | | |
| Methacrylic Acid Copolymer | 17.88 | 17.88 | 17.88 | Enteric Polymer |
| Dispersion | | | | |
| Triethyl Citrate | 1.79 | 1.79 | 1.79 | Plasticiser |
| Glycerol Monostearate | 0.89 | 0.89 | 0.89 | Anti-adherent |
| Polysorbate 80 | 0.36 | 0.36 | 0.36 | Surfactant |
| Sterile Water for Irrigation | q.s. | q.s. | q.s. | Process aid |
| Total Unit Weight | 477.05 | 477.05 | 477.05 | |

### Example 2: Composition of GSK2982772 Modified Release Coated Tablets, 60 mg and 480 mg

| | Quantity (mg) per Tablet | | | | | |
|---|---|---|---|---|---|---|
| | 60 mg | 60 mg 50:50 | 60 mg | 480 mg | 480 mg 50:50 | 480 mg |
| Component | 100% K100 | K100:K4M | 100% K4M | 100% K100 | K100:K4M | 100% K4M |

Tablet Core

| | | | | | | |
|---|---|---|---|---|---|---|
| GSK2982772A | 60.00 | 60.00 | 60.00 | 480.00 | 480.00 | 480.00 |
| Hypromellose 2208 K100LV | 178.00 | 89.0 | — | 178.00 | 89 | — |
| Hypromellose 2208 K4M | — | 89.0 | 178.00 | — | 89 | 178.00 |
| Lactose monohydrate | 562.00 | 562.00 | 562.00 | 142.00 | 142.00 | 142.00 |
| Sterile Water for Irrigation | q.s. | q.s. | q.s. | q.s. | q.s. | q.s. |
| Lactose anhydrous | 81.10 | 81.10 | 81.10 | 81.10 | 81.10 | 81.10 |
| Magnesium Stearate | 8.90 | 8.90 | 8.90 | 8.90 | 8.90 | 8.90 |
| Tablet Core Weight | 890.00 | 890.00 | 890.00 | 890.00 | 890.00 | 890.00 |

Sub Coat

| | | | | | | |
|---|---|---|---|---|---|---|
| Opadry ® Beige 02B170003 | 17.80 | 17.80 | 17.80 | 17.80 | 17.80 | 17.80 |
| Sterile Water for Irrigation | q.s. | q.s. | q.s. | q.s. | q.s. | q.s. |

Enteric Coat

| | | | | | | |
|---|---|---|---|---|---|---|
| Methacrylic Acid Copolymer | 27.38 | 27.38 | 27.38 | 27.38 | 27.38 | 27.38 |
| Dispersion | | | | | | |
| Triethyl Citrate | 2.74 | 2.74 | 2.74 | 2.74 | 2.74 | 2.74 |
| Glycerol Monostearate | 1.37 | 1.37 | 1.37 | 1.37 | 1.37 | 1.37 |
| Polysorbate 80 | 0.55 | 0.55 | 0.55 | 0.55 | 0.55 | 0.55 |
| Sterile Water for Irrigation | q.s. | q.s. | q.s. | q.s. | q.s. | q.s. |
| Total Unit Weight | 939.84 | 939.84 | 939.84 | 939.84 | 939.84 | 939.84 |

### Example 3: Composition of GSK2982772 480 mg DiffCORE Modified Release Coated Tablets

| | Quantity (mg) | |
|---|---|---|
| | per Tablet | |
| | 480 mg 25:75 | |
| Component | K100:K4M | Function |
| Tablet Core | | |
| GSK2982772A | 480.00 | Active |
| Hypromellose 2208 (K100LV) | 44.48 | Release Modifier |
| Hypromellose 2208 (K4M) | 133.52 | Release Modifier |
| Lactose monohydrate | 142.00 | Diluent |
| Sterile Water for Irrigation | q.s. | Granulating Fluid |
| Lactose anhydrous | 81.10 | Diluent |
| Magnesium Stearate | 8.90 | Lubricant |
| Tablet Core Weight | 890.00 | |
| Sub Coat | | |
| Opadry ® Beige 02B170003 | 17.80 | Film coat |
| Sterile Water for Irrigation | q.s. | Process aid |
| Enteric Coat | | |
| Methacrylic Acid Copolymer | 27.38 | Enteric Polymer |
| Dispersion | | |
| Triethyl Citrate | 2.74 | Plasticiser |
| Glycerol Monostearate | 1.37 | Anti-adherent |
| Polysorbate 80 | 0.55 | Surfactant |
| Sterile Water for Irrigation | q.s. | Process aid |
| Total Unit Weight | 939.84 | |

### Example 4: Composition of GSK2982772 Modified Release Tablet, 240 mg

| | | | |
|---|---|---|---|
| | Quantity | | |
| | per Tablet | | |
| Component | (mg) | Function | Reference to Standard |

Intra-Granular Composition

| | | | |
|---|---|---|---|
| GSK2982772A¹ | 240.0 | Active | Drug Substance |
| | | Pharmaceutical | Section |
| | | Ingredient | |
| Hypromellose | 120.0 | Release | Ph. Eur., USP |
| 2208 | | | |
| (K100LV) | | Modifier | |
| Hypromellose | 40.0 | Release | Ph. Eur., USP |
| 2208 | | | |
| (K4M) | | Modifier | |
| Lactose | 80.0 | Filler | Ph. Eur., USP-NF |
| Monohydrate | | | |
| Sterile Water for | q.s. | Granulation | Complies with Ph. Eur. |
| Irrigation² | | Fluid | Preparations for |
| | | | Irrigation (1116) |
| | | | and is manufactured |
| | | | with water that |
| | | | complies with |
| | | | Ph. Eur. Water |
| | | | for Injections in |
| | | | Bulk (0169) |
| Total per | 480.0 | — | — |
| Intra-Granule | | | |
| Lactose | 48.7 | Filler | Ph. Eur., USP-NF |
| (Anhydrous) | | | |
| Magnesium | 5.3 | Lubricant | Ph. Eur., USP-NF |
| Stearate | | | |
| Total per Tablet | 534.0 | — | — |

¹Quantity may be adjusted for potency factor of drug substance batch
²Removed during manufacturing process
q.s. = quantum satis (sufficient quantity)

### Example 5: Protocol for Clinical Trial 205017 (IR Vs. Minitabs MR): A Study to Compare the Pharmacokinetics (PK) of GSK2982772 Following Administration of Different Modified Release (MR) Formulations in Capsule and MR Tablet Formulations Relative to an Immediate Release (IR) Tablet Formulation and to Check the PK of MR Formulation in Capsule Following Repeat Doses

A three part, non-randomized, open label study designed to assess the pharmacokinetics of GSK2982772 following administration of mini-tab modified release formulations in a capsule relative to an immediate release reference tablet formulation (Part A), the pharmacokinetics of escalating, repeat doses of a selected mini-tab modified release prototype (Part B), and the pharmacokinetics of GSK2982772 following administration of modified release tablet formulations in the fed and fasted state (Part C) in healthy participants.

This was an open label, 3-part, single and repeat dose study conducted in healthy participants to assess modified release (MR) minitablets (MD in a capsule and MR monolithic matrix (MM) formulations of GSK2982772 compared to immediate release (IR) tablet formulation of GSK2982772.

Main Objective of the trial: To evaluate how much of the test medicine is taken up by the body after single doses of test formulations (120 mg) compared to reference formulation (120 mg). To evaluate how much of the test medicine is taken up by the body following a single dose of the MR tablet (240 mg) compare to the IR formulation (240 mg).

Arm Title: MT-12 hr Fasted/MT-8 hr Fasted/IR Fasted/MT-12 hr Fed (standard) GSK2982772 MR was available as prototype MR minitablet in capsules with unit dose strength of 60 mg in Part A. Participants in Part A received a single dose of 120 mg GSK2982772 MR MT-12 hour (hr) capsule (80% release at 12 hours) in fasted state in Period 1 followed by a single dose of 120 mg GSK2982772 MR MT-8-hour capsule (80% release at 8 hours) in fasted state in Period 2. In Period 3, participants received a single oral dose of 120 milligram (mg) GSK2982772 (4×30 mg) IR tablet in fasted state followed by a single oral dose of 120 mg GSK2982772 MR MT-12 hour capsule (80% release at 12 hours) after high fat meal in Period 4. There was a washout period of 7 days between each treatment period. All doses were administered via the oral route with 240 milliliters (mL) of water.

In part A, GSK2982772 IR tablet was available with unit dose strength of 30 mg and the total dose administered by participants was 120 mg (4 tablets of dose strength 30 mg) orally with 240 mL of water.

In Part B, GSK2982772 Minitab MR in capsules with unit dose strength of 15, 30 or 60 mg was administered by participants for Days 1 to 3. Participants in Part B received once daily dose of 120 mg GSK2982772 MR MT-12 hour capsule (80% release at 12 hours) in fasted state for 3 days in Period 1 followed by once daily dose of 240 mg GSK2982772 MR MT-12 hour capsule (80% release at 12 hours) in fasted state for 3 days in Period 2. In Period 3, participants received once daily dose of 300 mg GSK2982772 MR MT-12 hour capsule (80% release at 12 hours) in fed state (standard meal) for 3 days. There was washout period of 7 days between each treatment period. All doses were administered via oral route with 240 mL water.

Arm Title: MM240 Fast/IR240 Fast/MM480 Fast/MM480 Fed/MM480 DF/MM240 DF

In Part C, GSK2982772 MR tablet with unit dose strength of 240, 360 and 480 mg was administered by participants. Participants in Part C received a single dose of 240 mg GSK2982772 MR MM-12 hour tablet (80% release at 12 hours) in fasted (Fast) state in Period 1 followed by a single dose of 240 mg GSK2982772 IR tablet in fasted state in Period 2. In Period 3, participants received a single dose of 480 mg GSK2982772 MR MM-12-hour tablet (80% release at 12 hours) in fasted state. In Period 4, participants received a single dose of 480 mg GSK2982772 MR MM-12-hour tablet (80% release at 12 hours) in fed state followed by a single dose of 480 mg GSK2982772 MR MM-12 hours (80% release at 12 hours) before standard breakfast (delayed fed [DF]) in Period 5. In Period 6, participants received a single dose of 240 mg GSK2982772 MR MM-12 hours (80% release at 12 hours) before high-fat standard breakfast (DF). There was a washout period of 7 days between each treatment period. All doses were administered via oral route with 240 mL water

In part C, GSK2982772 IR tablet will be available with unit dose strength of 30 mg and the total dose administered by subjects was 240 mg (8 tablets of dose strength 30 mg) orally with 240 mL of water.

### Example 6: A Two-Part, Non-Randomized, Open Label Study Designed to Assess the Pharmacokinetic Profile of Modified Release Prototype Coated Tablet Formulations of GSK2982772 Relative to an Immediate Release Reference Tablet Formulation at a Fixed Strength (Part A) and the Pharmacokinetic Profile of Alternative Tablet Strengths of the Selected Modified Release Prototype Coated Tablet Formulation (Part B, Optional) in Healthy Participants

This was an open label, non-randomised single centre, two-part, single dose study in healthy male and female participants to assess modified release (MR) prototype coated tablet formulations of GSK2982772. This was the second study to evaluate MR prototypes. The first study (205017) assessed MR prototypes as minitabs in capsules and matrix monolithic tablet formulations. The current study evaluated DiffCORE prototype formulations with different release rates of GSK2982772. The impact of food (High-Fat breakfast) on the rate and extent of absorption of GSK2982772 was evaluated for selected DiffCORE MR formulations, and an assessment of alternative tablet strengths and dosing of multiple dose units was also performed. An assessment of dosing following a standard breakfast was also included.

Part A of the study was a non-randomized 6 period, 6-way fixed sequence design in which 3 DiffCORE MR prototype formulations were evaluated following single dose administration in the fasted or fed state (240 mg). Periods 1, 2 and 3 evaluated MR1 (target 80% release in 12 h), immediate release (IR) tablet (240 mg) and MR2 (target 80% release in 18 h), respectively in the fasted state. There were interim decisions after Periods 3, 4 and 5 to determine the formulation and prandial status to be investigated in the next period. In Periods 4 and 5, MR2 (target 80% release in 18 h) and MR1 (target 80% release in 12 h), respectively, were dosed following a High-Fat breakfast. In Period 6, MR3 formulation (target 80% release in 16 h) was dosed in the fasted state. Following completion of Part A, there was an interim review to determine whether to proceed with optional Part B. It was decided to proceed with Part B using MR3 (target 80% release in 16 h) formulation.

Part B of the study was an open-label, non-randomized up to 7-period fixed sequence study design to evaluate MR3 (80% release in 16 h) at different tablet strengths or as multiple unit doses. The tablet strengths selected in Part B were based on the exposure of GSK2982772 achieved in a high dose pharmacokinetic study in which doses of the IR tablet up to 240 mg three times daily (TID) were being administered, and the bioavailability of the selected MR prototype coated tablet formulations relative to the IR tablet (from Part A). Following review of the data from Part A, three different dose levels (120 mg, 480 mg and 960 mg) were selected for administration in Part B. In Part B, administration of the MR prototype coated tablet formulations were either in the fed or fasted state. Periods 1 and 2 evaluated dose levels of 480 mg and 960 mg, respectively in the fasted state. Periods 3 and 4 evaluated 480 mg in the fed state (High Fat Meal) and 120 mg in the fasted state, respectively. Period 5 evaluated the 480 mg enteric coated formulation in the fed state (High-Fat meal) and Period 6 evaluated the 480 mg formulation in the fed state (standard meal). There was an interim review following each of Periods 1 to 5 of Part B which determined the dose level, formulation and prandial status, as appropriate, for each subsequent period. Optional Period 7 was not conducted.

For Part A, an evaluable participant had received the IR tablet formulation (reference) and at least one of the MR formulations and completed the planned safety and PK assessments up to 24 h after dosing. For the food effect assessment, an evaluable participant had received the chosen MR formulation in the fasted state and the relevant prandial state and completed the planned safety and PK assessments up to 48 h after dosing. For Part B, an evaluable participant had completed the planned safety and PK assessments up to 48 h after dosing with at least one MR prototype coated tablet formulation.

All PK analyses were performed on the PK Population.

For all parts of the study, plasma GSK2982772 concentration-time data were analyzed by non-compartmental methods using WinNonlin v8.0.

### Example 7: A Multicenter, Randomized, Double-Blind, Placebo-Controlled Study to Evaluate the Benefit and Safety of GSK2982772 in Moderate to Severe Psoriasis Participants

Purpose: Plaque psoriasis is a chronic relapsing inflammatory skin disease that is characterized by keratinocyte hyper-proliferation and epidermal hyperplasia. Standard treatment for psoriasis generally requires long-term use of topical therapies, psoralen and ultraviolet A (PUVA), ultraviolet B (UVB) and/or systemic immunosuppressant therapies to achieve and maintain adequate disease control. This is a multicenter, randomized, double-blind study conducted in participants with moderate to severe plaque psoriasis. The study will evaluate the efficacy, safety, pharmacokinetic and pharmacodynamics profile of 960 milligram (mg) GSK2982772 administered as a once daily modified release (MR) formulation. Participants will be randomized in a 2:1 ratio to receive either 960 mg GSK2982772 or placebo for 12 weeks. The duration of the study, including Screening and follow-up, will be approximately 21 weeks for each participant.

### Primary Outcome Measures:

Percentage of participants who achieve >=75 percent improvement from Baseline in Psoriasis Area Severity Index (PASI) score at Week 12 [Time Frame: Baseline and Week 12]

Psoriatic lesions will be assessed using the PASI scoring system. Erythema, induration, and scale are each graded on a 5-point scale (0-4), and the percent body Surface Area (BSA) affected is scored on a 7-point scale (0-6) for each of the 4 specified body regions (head, upper extremities, trunk, and lower extremities). The body region scores are each multiplied by a weighted factor; and the sum of the region scores give the overall PASI score. Higher scores indicate more severe disease.

Secondary Outcome Measures:
Percentage of participants who achieve >=50 percent improvement from Baseline in PASI score at Week 12 [Time Frame: Baseline and Week 12]

Psoriatic lesions will be assessed using the PASI scoring system. Erythema, induration, and scale are each graded on a 5-point scale (0-4), and the percent BSA affected is scored on a 7-point scale (0-6) for each of the 4 specified body regions (head, upper extremities, trunk, and lower extremities). The body region scores are each multiplied by a weighted factor; and the sum of the region scores give the overall PASI score. Higher scores indicate more severe disease.
Percentage of participants who achieve >=90 percent improvement from Baseline in PASI score at Week 12 [Time Frame: Baseline and Week 12]
   Psoriatic lesions will be assessed using the PASI scoring system. Erythema, induration, and scale are each graded on a 5-point scale (0-4), and the percent BSA affected is scored on a 7-point scale (0-6) for each of the 4 specified body regions (head, upper extremities, trunk, and lower extremities). The body region scores are each multiplied by a weighted factor; and the sum of the region scores give the overall PASI score. Higher scores indicate more severe disease
Percentage of participants who achieved >=100 percent improvement from Baseline in PASI score at Week 12 [Time Frame: Baseline and Week 12]
   Psoriatic lesions will be assessed using the PASI scoring system. Erythema, induration, and scale are each graded on a 5-point scale (0-4), and the percent BSA affected is scored on a 7-point scale (0-6) for each of the 4 specified body regions (head, upper extremities, trunk, and lower extremities). The body region scores are each multiplied by a weighted factor; and the sum of the region scores give the overall PASI score. Higher scores indicate more severe disease
Change from Baseline PASI scores at Week 12 [Time Frame: Baseline and Week 12]

Psoriatic lesions will be assessed using the PASI scoring system. Erythema, induration, and scale are each graded on a 5-point scale (0-4), and the percent BSA affected is scored on a 7-point scale (0-6) for each of the 4 specified body regions (head, upper extremities, trunk, and lower extremities). The body region scores are each multiplied by a weighted factor; and the sum of the region scores give the overall PASI score. Higher scores indicate more severe disease.
Percentage of participants who have a Static Investigator's Global Assessment (sIGA) score of 0 or 1 at Week 12 [Time Frame: At Week 12]

The Investigator or physician designee only will complete a global assessment of disease activity using the physician global assessment item. A 5-point scoring system will be used to measure the severity of psoriatic lesions over the entire body at the time of evaluation. Percentage of participants who have a sIGA score of 0=clear or 1=almost clear at Week 12 will be summarized.
Change from Baseline in psoriatic BSA at Week 12 [Time Frame: Baseline and Week 12]

The BSA affected with psoriasis will be evaluated at all study visits by the Investigator or suitably trained delegate. As a reference, the area of the whole palm is counted as 1 percent BSA. Estimated Enrollment: 21

### Arms Assigned Interventions

Experimental: Participants receiving GSK2982772 960 mg
Participants will receive GSK2982772 960 mg oral tablets once daily for 12 weeks.

### Drug: GSK2982772

GSK2982772 will be available as MR tablet at a unit dose strength of 480 mg.
Placebo Comparator: Participants receiving placebo
Participants will receive GSK2982772 matching placebo oral tablets once daily for 12 weeks.

### Drug: Placebo

GSK2982772 matching placebo tablets will be administered via the oral route.
Example 8: Manufacturing Process of GSK2982772 Modified Release Prototype Coated Tablets, 240 mg and Controls

It is to be understood that the invention is not limited to the aspects or embodiments illustrated hereinabove and the right is reserved to the illustrated aspects or embodiments and all modifications coming within the scope of the following claims.

1. A modified release pharmaceutical composition comprising an erodible matrix core, which core comprises a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, or a salt thereof.

2. The modified release pharmaceutical composition according to claim 1, wherein the erodible matrix core further comprises at least one component which is a release modifier.

3. The modified release pharmaceutical composition according to claim 2, wherein the erodible matrix core has a film coating around said core.

4. The modified release pharmaceutical composition according to claim 3, wherein the film coating comprises at least one erodible material and at least one aperture in the film coating.

5. The modified release pharmaceutical composition according to claim 4, wherein the at least one aperture in the film coating is of pre-determined size.

6. The modified release pharmaceutical composition according to claim 4, wherein the at least one aperture creates an exposed surface area in the film coating.

7. The modified release pharmaceutical composition according to claim 4, wherein the aperture is about 4.0 mm to about 5.5 mm.

8. A modified release pharmaceutical composition according to claim 1 comprising an erodible matrix core, which core comprises 480 mg of the compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof.

9. The modified release pharmaceutical composition according to claim 1, which is in the form of a tablet.

10. A tablet comprising an erodible matrix core, which core comprises:
(a) about 30 mg to about 480 mg of a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, or a salt thereof;
(b) about 1.0% wt/wt to about 60% wt/wt of a release modifier;
(c) about 1.0% wt/wt % to about 95% wt/wt of a diluent;
(d) about 0.25% wt/wt to about 5.0% wt/wt of a lubricant; and
(e) a film coating around said erodible matrix core comprising an erodible material in an amount of about 1.0% wt/wt of the tablet core and about 10% wt/wt of the tablet core, and at least one aperture in the film coating.

11. A tablet comprising an erodible matrix core, which core comprises:
(a) about 240 mg to about 480 mg of a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, or a salt thereof;
(b) about 1.0% wt/wt to about 20% wt/wt of a release modifier;
(c) about 15% wt/wt % to about 30% wt/wt of a diluent;
(d) about 0.50% wt/wt to about 3.0% wt/wt of a lubricant; and
(e) a film coating around said erodible matrix core comprising an erodible material in an amount of about 2.0% wt/wt of the tablet core and about 6% wt/wt of the tablet core, and at least one aperture in the film coating.

12. A tablet comprising an erodible matrix core, which core comprises:
(a) about 480 mg of a compound which is (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, or a salt thereof;
(b) about 20% wt/wt of a release modifier;
(c) about 25% wt/wt of a diluent;
(d) about 1.0% wt/wt of a lubricant; and
(e) a film coating around said erodible matrix core comprising an erodible material in an amount of about 4% wt/wt of the tablet core, and at least one aperture in the film coating.

13. The tablet according to claim 1, wherein the tablet is suitable for once daily dosing.

14. The modified release composition according to claim 2, wherein the release modifier is selected from cross-linked sodium carboxymethylcellulose, cross-linked hydroxypropylcellulose, hydroxyethylcellulose, high-molecular weight hydroxypropylmethylcellulose, carboxymethyl amide, potassium methacrylate divinylbenzene co-polymer, polymethyl-methacrylate, cross-linked polyvinylpyrrolidone, hydroxyethyl cellulose, high-molecular weight polyvinyl alcohols.

15. A modified release tablet according to claim 1 comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, wherein, upon administration to a human, produces a maximum blood plasma concentration of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, which is about 70% to about 90% lower when compared to the maximum blood plasma concentration of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, produced by an instant release tablet.

16. A modified release tablet according to claim 1 comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, wherein, upon administration to a human, produces a total drug exposure across time value of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, which is about 30% to about 55% lower when compared to the total drug exposure across time value of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, produced by an instant release tablet.

17. A modified release tablet according to claim 1 comprising a crystalline form of (S)-5-benzyl-N-(5-methyl-4-oxo-2,3,4,5-tetrahydrobenzo [b][1,4]oxazepin-3-yl)-4H-1,2,4-triazole-3-carboxamide, or a tautomer thereof, wherein, upon administration to a human, produces a blood plasma concentration at 24 hours of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, which is about 10-fold to about 25-fold lower when compared to the blood plasma concentration at 24 hours of (S)-5-benzyl-N-(5-methyl-4-oxo-2,3,4,5-tetrahydrobenzo [b][1,4]oxazepin-3-yl)-4H-1,2,4-triazole-3-carboxamide, or a tautomer thereof, produced by an instant release tablet.

18. A modified release tablet according to claim 1 comprising a crystalline form of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, wherein, upon administration to a human, produces a time to maximum concentration value of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, which is about 3 to about 4 hours greater than the time to maximum concentration value of (S)-5-benzyl-N-(5-methyl-4-oxo-2,3,4,5-tetrahydrobenzo[b][1,4]oxazepin-3-yl)-4H-1,2,4-triazole-3-carboxamide, or a tautomer thereof, produced by an instant release tablet.

19. A method of treating a subject with psoriasis comprising administering to said subject the modified release composition according to claim 1.

20. The method of claim 19, wherein said subject is a subject with moderate to severe psoriasis.
